(19) United States
(12) Patent Application Publication
(10) Pub. No.: US 2008/0281298 A1
(43) Pub. Date: Nov. 13, 2008

(54) Electronic support system for biological data sensor

(76) Inventors: David R. Andersen, Coralville, IA (US); Kiran Kanukurthy, Iowa City, IA (US); Jason Wu, West Des Moines, IA (US)

Correspondence Address: Ballard Spahr Andrews & Ingersoll, LLP, SUITE 1000, 999 PEACHTREE STREET, ATLANTA, GA, 30309-3915, US

(21) Appl. No.: 11/348,615

(22) Filed: Feb. 7, 2006

Related U.S. Application Data
(60) Provisional application No. 60/650,678, filed on Feb. 7, 2005.
(60) Provisional application No. 60/667,973, filed on Apr. 4, 2005.

(51) Int. Cl. A61K 9/22; A61B 5/055

(52) U.S. Cl. 604/891.1; 600/347

(57) ABSTRACT

An electronic support system for controlling a biological data sensor and related methods of use are disclosed herein.

### CROSS REFERENCE TO RELATED APPLICATIONS

This application claims priority to U.S. Provisional Patent Application Ser. No. 60/650,678 filed in the United State Patent and Trademark Office on Feb. 7, 2005, and to U.S. Provisional Patent Application Ser. No. 60/667,973 filed in the United State Patent and Trademark Office on Apr. 4, 2005. The entire disclosures of these applications are hereby incorporated by reference in their entirety for all purposes.

The invention described in the foregoing specification has been developed in part with funds received from the National Institutes of Health under grant number DK-64569 The United States Government may have certain rights under this invention.

### FIELD OF THE INVENTION

The present invention relates generally to the field of biological data sensors and more particularly to an electronic support system for use in connection with a biological data sensor.

### BACKGROUND OF THE INVENTION

Diabetes is a chronic, incurable disease that causes an array of serious medical complications and premature death. Complications include heart disease, stroke, kidney failure, and nervous system disorders. Although diabetes is a potentially devastating disease, early diagnosis and tight glycemic control can greatly diminish the medical complications and cost of this disease.

The goal of tight control is to maintain one's blood glucose levels within a physiologically acceptable range. Tight control therefore typically requires frequent blood glucose measurements, which provides the information needed to administer insulin or glucose properly. The pain, cost and inconvenience of state-of-the-art glucose monitoring technology impede frequent monitoring and are primarily responsible for the failure of patients to maintain tight control. Thus, it has been recognized for several decades that an ideal treatment of diabetes would involve a closed-loop insulin delivery system that is implanted within the patient's body.

This so-called artificial pancreas could comprise an insulin delivery pump coupled with some type of glucose-sensing technology. Using this system, insulin could be delivered continuously in response to detected changes in the blood glucose concentrations. However, for this to system to be operable, the glucose sensing component must be able to provide accurate and rapid blood glucose values to a micro-processing unit, which would compute the amount of insulin required and then control the required insulin delivery. Accordingly, the successful development of an artificial pancreas or other artificial biological delivery system as described above depends on the development of implantable analyte (i.e., glucose) sensing technology and corresponding electronic support that can reliably control the instrumentation. Thus, there is a need in the art for implantable analyte sensing technology and electronic support that can enable the continuous operation of an analyte sensor for extended durations with minimal or even no user intervention required.

### SUMMARY OF THE INVENTION

The present invention is based, in part, upon the invention of an electronic support system that can, in one aspect, be integrated with a biological data sensor and can enable the continuous operation of the data sensor for extended durations with minimal or even no user intervention required.

In one aspect, the present invention provides an electronic support system for controlling a biological data sensor. The electronic support system can comprise a primary module, comprised of a main controller having a data acquisition unit interface and a telemetry unit interface. A data acquisition unit can be provided in communication with the main controller through the data acquisition unit interface and can be configured to receive data transmitted from a biological data sensor. A first telemetry unit can also be provided in communication with the main controller through the telemetry unit interface and can be configured to communicate with a second or remote telemetry unit. An internal power unit can also be provided for powering one or more components of the primary module. The support system can further comprise a remote module external to the primary module. The remote module can comprise a remote charging unit and/or a second telemetry unit.

In another aspect, the present invention provides a method for monitoring desired biological data. For example, the method of the present invention can comprise the monitoring of a particular analyte concentration in a test subject. To this end, in one aspect, the method of the present invention can comprise sampling electromagnetic absorption data of a desired analyte in a test subject with an implantable analyte sensor, wherein the sampled data can be obtained in a first format. The sampled data in the first format can be transformed to sampled data in a second format and transmitted in the second format to a main controller unit wherein at least a portion the data can be stored and/or time stamped to create processed data. At least a portion of the processed data can be transmitted to a remote telemetry module from which the concentration of the analyte in the test subject can be determined for a predetermined time.

Additional aspects of the invention will be set forth, in part, in the detailed description, figures and any claims which follow, and in part will be derived from the detailed description, or may be learned by practice of the invention. It is to be understood that both the foregoing general description and the following detailed description are exemplary and explanatory only and are not restrictive of the invention as disclosed.

### BRIEF DESCRIPTION OF THE FIGURES

The accompanying drawings, which are incorporated in and constitute a part of this specification, illustrate several embodiments of the instant invention and together with the description, serve to explain, without limitation, the principles of the invention.

FIG. 1 illustrates an optical sensing element according to one aspect of the invention. As depicted, the optical sensing element comprises an array of 32 photodiodes.

FIG. 2 illustrates the near infrared absorption spectrum for glucose in the spectral range of from approximately 2.05 μm to approximately 2.4 μm.

FIG. 3 illustrates a functional block diagram of an exemplary electronic support unit according to one aspect of the present invention.

FIG. 4 is a schematic of an exemplary main controller unit according to one aspect of the present invention.

FIG. 5 illustrates an exemplary data acquisition from one photodiode of the photodiode array depicted in FIG. 1.

FIG. 6 is an exemplary schematic of a data acquisition unit according to one aspects of the present invention.

FIG. 7 is an exemplary schematic of a data acquisition unit according to one aspect of the present invention.

FIG. 8 is a graphical illustration of exemplary digitized data obtained from a data acquisition unit according to one aspect of the present invention. Error bars on the diagram indicate the standard deviation of the noise on the data.

FIG. 9 is a graphical illustration of the exemplary digitized data obtained from a data acquisition unit according to one aspect of the present invention, wherein the signal to noise ratio of the data has been increased by the use of a digital filtering algorithm. No error bars are shown because their width is insignificant on the scale of the drawing.

FIG. 10 is an exemplary schematic of a remote charger unit according to one aspect of the present invention.

FIG. 11 is an exemplary schematic diagram of an internal power unit according to one aspect of the present invention.

FIG. 12 illustrates a flow chart diagram of a battery charging cycle according to one aspect of the present invention.

FIG. 13 illustrates an exemplary schematic diagram of an internal telemetry unit according to one aspect of the present invention.

FIG. 14 illustrates an exemplary schematic diagram of an external telemetry unit according to one aspect of the present invention.

FIG. 15 illustrates an exemplary physical implantation and use of an electronic support unit and analyte sensor according to the present invention.

FIG. 16 illustrates an exemplary schematic diagram of an internal module according to an alternative aspect of the present invention.

FIG. 17 illustrates an exemplary schematic diagram of an external module according to an alternative aspect of the present invention.

### DETAILED DESCRIPTION OF THE INVENTION

The present invention may be understood more readily by reference to the following detailed description, and figures, and their previous and following description.

Before the present compositions, devices, and/or methods are disclosed and described, it is to be understood that this invention is not limited to the specific articles, devices, and/or methods disclosed unless otherwise specified, as such may, of course, vary. It is also to be understood that the terminology used herein is for the purpose of describing particular aspects only and is not intended to be limiting.

As used herein, the singular forms “a,” “an” and “the” include plural referents unless the context clearly dictates otherwise. Thus, for example, reference to an “analyte” includes aspects having two or more such analytes unless the context clearly indicates otherwise.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

As briefly stated above, in one aspect the present invention provides an electronic support system, also referred to herein as an electronic support unit (ESU), for use in connection with a biological data sensor, such as for example, an implantable analyte sensor. In one aspect the electronic support system is in communication with an analyte sensor and can enable the continuous and reagent-free optical analysis of interstitial fluid (ISF) present within a test subject. To this end, in one aspect, the electronic support system can provide a physical interface between one or more optical sensing elements in an analyte sensor and obtained analyte related data.

The electronic support system of the present invention can be used in connection with any suitable analyte sensor and therefore is not limited to the exemplary analyte sensors disclosed herein. Thus, in one aspect, a suitable analyte sensor can comprise any number of conventional components that together are capable of irradiating interstitial fluid from a test subject with electromagnetic radiation and subsequently detecting variations in the electromagnetic radiation resulting at least from the interface of the electromagnetic radiation with the interstitial fluid. In one aspect, the analyte sensor can be implantable in the subcutaneous tissue of a test subject. In another aspect, the analyte sensor can comprise a light source, such as a broadband LED, for providing electromagnetic radiation in a desired band of wavelengths and at a desired level of intensity. The sensor can further comprise an optical sampling chamber and a spatially variable wavelength filter in communication with an array of optical sensing elements, also referred to herein as photodetector elements.

In use, an exemplary analyte sensor can be implanted in the subcutaneous tissue of a test subject. According to this aspect, the analyte sensor can be constructed and arranged such that sampled interstitial fluid can enter and exit an optical sampling chamber by way of, for example, two micro-channels constructed and arranged in the central region of the optical sampling chamber. Thus, electromagnetic radiation can pass from the light source, such as an LED, through the optical sampling chamber, through the spatially variable wavelength filter and then be detected by an array of photodetector elements. One of skill in the art will appreciate that an analyte sensor according to this exemplary aspect does not require the use of moving or adjustable parts and can therefore occupy a relatively small volume of space. Thus, an analyte sensor according to this aspect can occupy as small or as large a volume as is desired. For example, the analyte sensor can occupy a volume as small as the technology of the individual components themselves will allow. In one aspect, and without limitation, an analyte sensor according to the present invention can occupy a volume in the range of from approximately 0.01 cm³ to approximately 1.0 cm³, including volumes of 0.02, 0.03, 0.04, 0.05, 0.06, 0.07, 0.08, 0.09, 0.1, 0.2, 0.3, 0.4, 0.5, 0.6, 0.7, 0.8, 0.9, and any range derived from these values. In still another aspect, the analyte sensor occupies a volume that does not exceed approximately 0.1 cm³.

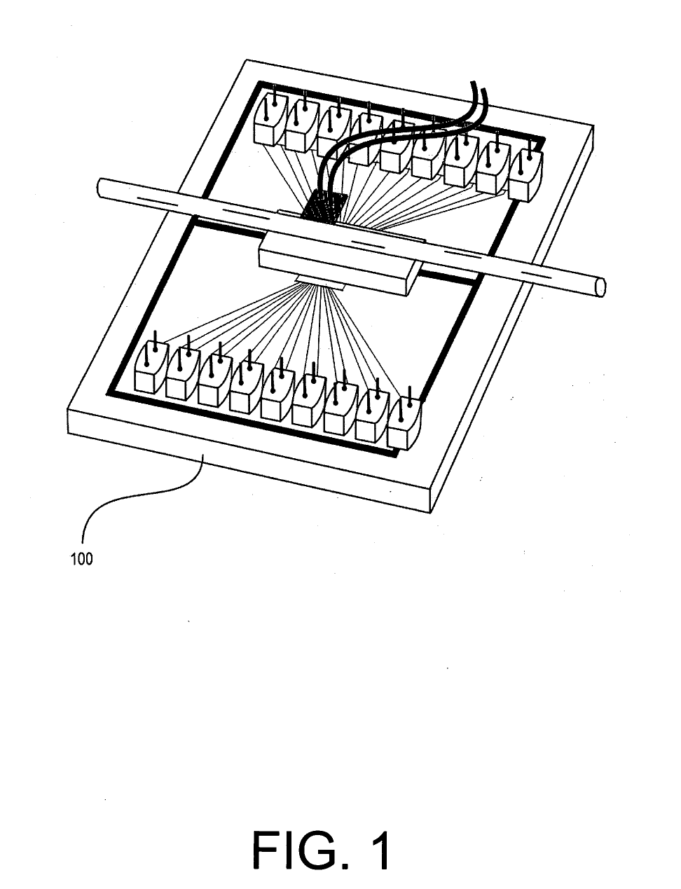

As one of ordinary skill in the art will appreciate, there are several conventional methods that can be used for performing spectrally resolved measurements on electromagnetic radiation that passes through an optical sampling chamber of an analyte sensor, including, without limitation, Fourier transform and dispersive techniques (utilizing diffraction gratings or dispersive prisms). While any of these methods can be used in connection with the present invention, in an alternative aspect, an analyte sensor 100 according to the present invention can provide a spectrally resolved measurement using a bandpass filter mounted on a photodiode array such as that depicted in FIG. 1. To this end, light exiting the optical sampling chamber will be incident on the bandpass filter and the filter can be configured such that the central wavelength of the passband varies along one of the dimensions of the filter. Thus, each detector element, or photodiode, can be sensitive to a different wavelength. The spectral resolution can then be determined by the width of the passband at each point, and the spectral point spacing can be determined by the number of detector array elements. As one of skill in the art will appreciate upon practicing the present invention, unlike conventional diffraction-based instruments, this method does not require the use of imaging optics. Accordingly, the bandpass filter and detector assembly can be mounted directly on the output of the optical sampling chamber.

It will also be appreciated that direct in situ sampling of interstitial fluid can simplify the task of detecting an analyte as compared to other conventional non-invasive measurement approaches that rely on detecting an analyte based on spectral data obtained from a more complex and/or heterogeneous skin matrix. More specifically, interstitial fluid is typically a clear fluid with relatively few or even no scattering particles (such as cells), and thus the optical throughput can be orders of magnitude higher than transmission measurements through skin or whole blood. Further, because the optical geometry of the method set forth above can be defined by the path length of the sampling chamber, the interpretation of measured spectra in terms of absolute analyte content can also be much more straight forward than for methods that rely on diffuse reflection or transflection arrangements.

The present invention is also not limited to its use in connection with any one particular test subject or group of test subjects. To this end, in one aspect, the test subject can be any living organism in which an analyte sensor as described herein can be implanted into the subcutaneous tissue thereof. For example, in one aspect, the test subject can be a plant. Alternatively, in another aspect, the test subject can be an animal. In one aspect the animal can be mammalian. In an alternative aspect the animal can be non-mammalian. The animal can also be a cold-blooded animal, such as a fish, a reptile, or an amphibian. Alternatively, the animal can be a warm-blooded animal, such as a human, a farm animal, a domestic animal, or even a laboratory animal.

The present invention is also not limited to any one particular analyte or group of analytes. To this end, in one aspect the analyte can be any physiological chemical having a functional group and/or chemical bond capable of providing an identifiable spectral signature or feature when irradiated by electromagnetic radiation, such as radiation in the near infrared (NIR) and/or middle infrared (MIR) wavebands. In one aspect, the functional group and/or chemical bond can be C-H, N-H, O-H, or any combination thereof. Specific and non-limiting examples of suitable analytes according to the instant invention include glucose, urea, lactate, triglyceride, protein, cholesterol, and ethanol. In one aspect, the analyte is glucose. In still another aspect, the analyte is urea.

It should also be understood that an analyte sensor according to the present invention can be configured to operate in the near infrared electromagnetic region, including radiation in the wave number range of from approximately 4000 cm⁻¹ to approximately 14500 cm⁻¹. To this end, the analyte sensor can be configured to operate in additional wave numbers of 5000, 5500, 6000, 6500, 7000, 7500, 8000, 8500, 9000, 9500, 10000, 10500, 11000, 11500, 12000, 12500, 13000, 13500 and 14000 cm⁻¹ and any range derived from these values. In still another aspect, and for example when used in an aqueous environment, like the human body, the analyte sensor can operate in the so-called combination spectral range of the near infrared spectrum over a wave number range from approximately 4000 cm⁻¹ to approximately 5000 cm⁻¹. As one of ordinary skill in the art will appreciate, spectral features in the combination spectral range originate from the combination of stretching and bending vibrational modes associated with C-H, O-H, and N-H chemical bonds within the molecules in the sample matrix. In still another aspect, and again for exemplary aqueous samples, the analyte sensor can operate in the so-called first overtone spectral region of the near infrared spectrum over the wave number range from approximately 5500 cm⁻¹ to approximately 6500 cm⁻¹. Spectral features in this first overtone spectral range correspond to the first overtone of C-H chemical bonds within these sample molecules.

In an alternative aspect, the analyte sensor can be configured to operate in the mid infrared electromagnetic region, including radiation in the wave number range from approximately 300 cm⁻¹ to approximately 4000 cm⁻¹. To this end, the analyte sensor can be configured to operate in additional sub-ranges within the wave number bands of 500, 1000, 1500, 2000, 2500, 3000, and 3500 cm⁻¹ and any range derived from these values. It should also be understood that for both near infrared and mid infrared analyte measurements, it is not required by the invention that the wavelength range used be a single contiguous range of wave numbers. For example, in still another aspect, a plurality of different segments of shorted wave number ranges can be used.

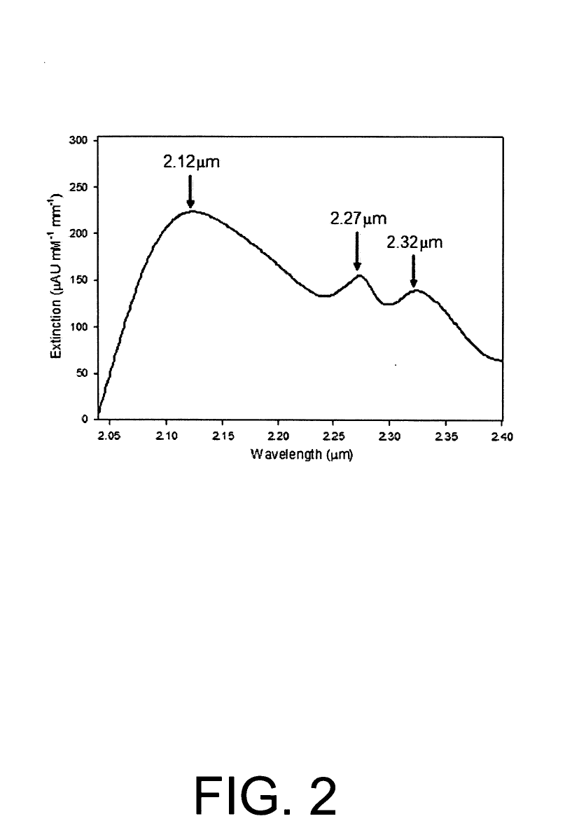

As one of ordinary skill in the art will appreciate, the desired operational waveband range of the analyte sensor will be dependent on the particular analyte under investigation. For example, in one aspect where the analyte is glucose, interstitial fluid from subcutaneous space is sampled through an embedded ultra filtration probe and subsequently enters into a micro fluidic chamber, physically isolated from the biological environment. The sample of interstitial fluid is then carried to an optimized spectrometer cell, where a 16 cm⁻¹ resolution near infrared spectrum can be collected over a spectral range of from approximately 4000 cm⁻¹ to approximately 5000 cm⁻¹ which corresponds to the spectral range containing a spectral signature unique to glucose, as depicted in FIG. 2. Using conventional mathematical models, the concentration of the glucose can be obtained from a direct analysis of the detected glucose absorption spectrum.

An electronic support system 200 according to the instant invention can be constructed and arranged so as to comprise a battery powered primary or internal module that can be affixed to a test subject and an external or remote module that can be positioned in a remote location a predetermined distance from the internal module. In one aspect, the primary or internal module can be optionally implanted in the subcutaneous tissue of a test subject. In an alternative aspect, the primary or internal module can be affixed to or worn on a surface of the test subject. For example, and without limitation, the primary module can be releasably affixed to or worn on the skin of a human test subject.

The electronic support system 200 can, in one aspect, enable continuous operation of a biological data sensor for extended durations with relatively minimal or even no user intervention. Further, the electronic support system 200 can operate from a battery based power supply capable of remote charging. To this end, the electronic support system 200 as configured and described herein can further operate at relatively low power supply voltages such as, for example, 3.3 volts. Such a power supply can provide continuous energy for up to and even exceeding 24 hours of system operability.

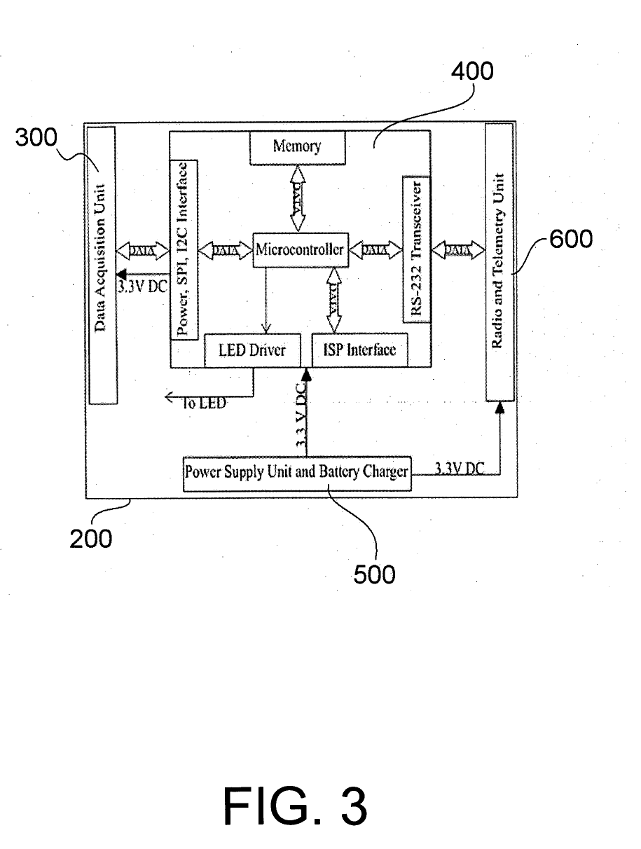

The primary or internal module comprises a data acquisition unit (DAU), a main controller unit (MCU) comprised of a dedicated microcontroller unit to control the sensor system; an internal power unit (IPU) to supply power to one or more of the components in the internal module, and a first telemetry unit (TU) for communicating analyte related data to the external or remote module. The external module can comprise a remote charger unit (RCU) that can transmit inductive power to the internal power unit, and a second telemetry unit that can receive sampled data that has been transmitted from the first telemetry unit of the internal module. Functionally, the ESU 200 in one aspect is therefor comprised of a Data Acquisition Unit (DAU) 300, Main Controller Unit (MCU) 400, Power Supply Unit (PSU) 500, and the Telemetry Unit (TU) 600 respectively, as shown in FIG. 3.

The main controller unit or MCU 400 can, in one aspect, provide one or more functions including, without limitation, obtaining sampled analyte data from the data acquisition unit, storing the obtained data in memory, packaging the data along with a time stamp, and/or subsequently transmitting the data through the telemetry unit (TU) 600. The main controller can also be responsible for coordinating the communication between the internal and external modules and ensuring proper operation of one or more units within the system.

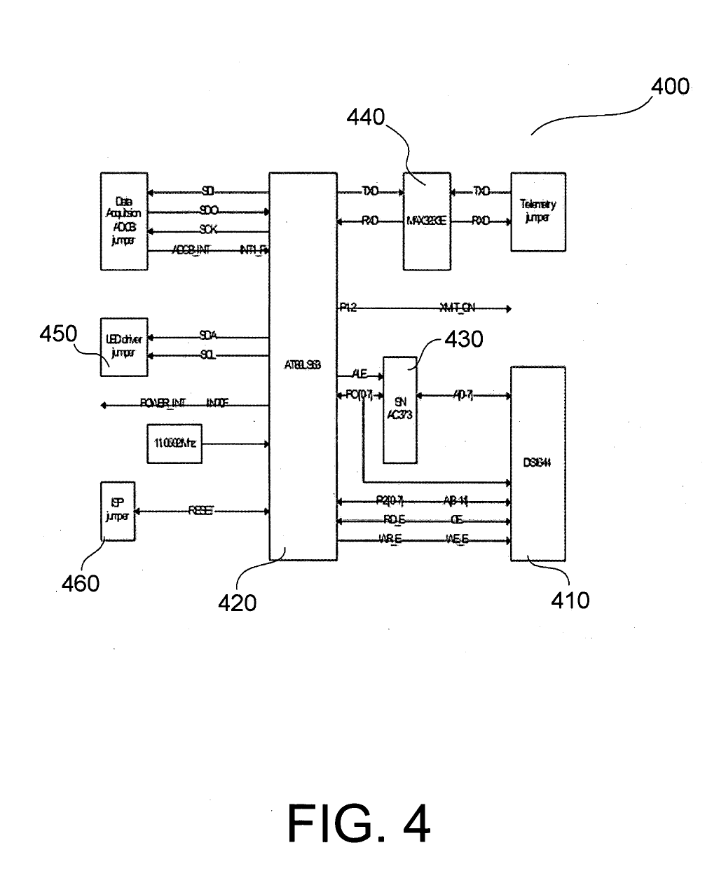

An exemplary schematic of an MCU 400 according to the instant invention is illustrated in FIG. 4. As depicted, the MCU comprises a memory component 410 and a microcontroller component 420. While any conventional memory device can be used with the MCU, the commercially available Dallas Semiconductor DS1644 NVRAM memory, equipped with a real time clock (RTC) and back-up Li-ion battery can be used for data storage in an exemplary aspect. As one of skill in the art will appreciate, the NVRAM with an integrated circuit can provide fast access to data and a real time clock for time-stamping the data. Further, the memory and real time clock combination can, in one aspect, eliminate the need for additional time keeping hardware. An alternative memory device which is suitable for use in the instant invention can be the Ramtron FM31256 32 KB FRAM memory, also equipped with a real time clock. The FRAM can offer virtually unlimited read/write cycles, relatively fast access to data, and as mentioned, a real time clock for time stamping the data.

In one aspect, it is desired for the memory capacity to be sufficient to store up to approximately 24 hours of sampled data. According to this aspect, at an exemplary sampling rate of one sample every 5 minutes and approximately 3 bytes of memory needed per sample per channel, an additional 6 bytes per sample for timestamp and error detection, a data memory capacity of at least 29.4 KB can be needed to store 24 hours of data obtained from a 32 photodiode array. To this end, one of skill in the art can appreciate that any desired memory capacity can be used in the instant invention and further, the desired memory capacity can be calculated according to the following equation:

Memory Capacity=d·R·S·n

where d is the duration in hours, R is the sampling rate in samples per hour; S is the sample size in bytes per channel per sample, n is the number of channels.

To enable data collection, light source control, data processing, and/or operation of the telemetry generation, a microcontroller 420 is incorporated into the Main Control Unit. According to this aspect, since the microcontroller can in one aspect be accessing data stored in external memory, a microcontroller that supports external memory can be used. To this end, as one of ordinary skill in the art will appreciate, it can also be desired, although not required, for a single microcontroller unit to support one or more of the other instrumental requirements, while maintaining as small of a size as possible with as low power consumption as possible.

In still another aspect, it can be further desired, although it is not required, for the microcontroller to comprise an instruction set supporting multiplication and division instructions such that it is capable of performing floating point operations. Additionally, a suitable microcontroller can comprise either an internal program flash or an external flash memory. Any conventional and commercially available microcontroller capable of performing one or more feature set forth above can be used in accordance with the present invention. However, the specific features described above can typically be provided in an exemplary conventional 8-bit microcontroller such as those tested and indicated in Table 1 below. While any one of the microcontrollers listed in Table 1 is suitable for use in the instant invention, a comparison of these four commercially available 8-bit microcontrollers indicates that in one aspect, a suitable microcontroller for use in the Main Controller Unit is the Atmel AT89C51ID2.

TABLE 1

| | Exemplary Microcontrollers | | | |
|---|---|---|---|---|
| Features | AT89LS53 | ATtiny26L | MC68HC805 | AT89C51ID2 |
| Architec- | 8051 | AVR | 68 | 8051 |
| ture | | | | |
| Supply | 2.7 V | 2.7 V | 5 V | 3.3 V |
| Voltage | | | | |
| Program | 12 kB | 2 kB | 8 kB | 64 kB |
| Memory | | | | |
| RAM | 256 | 128 | 192 | 2048 |
| (bytes) | | | | |
| IO Pins | 32 | 16 | 20 | 32 |
| Clock | 12 MHz | 16 MHz | 4 MHz | 160 MHz |
| Speed | | | | |
| (Max) | | | | |
| ISP | Yes | Yes | No | Yes |
| MUL, | Yes | No | No | Yes |
| DIV Inst | | | | |
| Interrupts | 9 | 11 | 10 | 9 |
| Timers | 3, 16-bit | 2, 8-bit | 16-bit, 8-bit | 3, 16-bit |
| UART | Yes | No | No | No |
| Module | | | | |
| SPI | Yes | No | No | No |
| Module | | | | |

In still another aspect, a suitable microcontroller can typically supply multiplexed address-data lines. Thus, in order to access the external memory, a transparent octal D-type latch 430 with tri-state outputs can be used. To this end, a suitable latch for the multiplexing can, in one aspect, have a latch switching delay that is negligible as compared to the memory access time, which is typically of the order of 120 ns for the DS1644 NVRAM memory described above. An exemplary D-type latch that is suitable for use in the instant invention is the Texas Instrument SN74AC373 octal D-type latch with a switching delay of approximately 15 ns.

The microcontroller unit is also provided with a telemetry unit interface 440 to interface the telemetry unit with the main controller. The telemetry unit interface can be any communication interface such as, for example, USB, serial, firewire, parallel, and the like In one aspect, the telemetry unit interface can comprise an RS-232 serial port. The RS-232 serial port can provide added debugging functionality as well. Virtually any conventional and commercially available RS-232 serial port can be used to provide the telemetry interface. While any transceiver known in the art can be used, in one aspect, a suitable RS-232 transceiver can provide true RS-232 signal levels with minimum board space, low power consumption, and suitable operating voltage. To this end, the Maxim MAX3233EWE dual RS-232 transceiver with internal charge pumps is a non-limiting example of a RS-232 transceiver that is suitable for use in the instant invention. The MAX3233E can operate in the voltage range of 3.0-3.6V DC with 1 uA supply current. Additionally, the MAX3233E is capable of entering into a sleep mode when either the RS-232 cable is disconnected or when the UART driving the transmitter inputs is inactive for more than 30 seconds. From the sleep mode, the MAX3233E can turn on again when it senses a valid transition at any transmitter or receiver input. As one of skill in the art will appreciate, this feature can help to conserve power in the system.

The microcontroller can further comprise one or more peripheral support interfaces such as, for example, a jumper for a light source connector 450, an ISP jumper 460, and other serial interfaces to facilitate connectivity of other controller modules and/or other system components.

As stated above, the electronic support system further comprises a data acquisition unit (DAU) 300 that can obtain sampled data from a biological data sensor, such as for example, data detected by the optical sensing component of an analyte sensor. In one aspect, the data acquisition unit can obtain data in a first format and can transform that sample data into a second format. For example, the DAU 300 can obtain sampled voltage data from a photodiode array in an analog format and can transform the analog data into digital format having a predetermined level of precision.

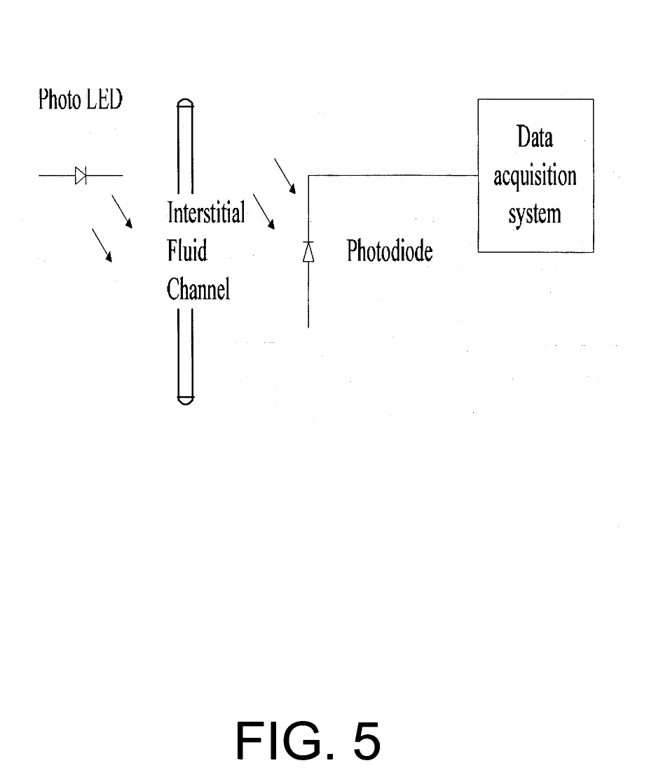

In one aspect, the data acquisition unit (DAU) 300 can comprise a current integrator and an analog/digital (A/D) converter. The A/D converter can be interfaced to the main controller unit through any conventional interface, such as for example a serial peripheral interface (SPI). The level of precision for A/D conversion can vary as desired and can in one aspect be in the range of from at least 8 bits up to and even exceeding 128 bits, including additional precision values of 16, 20, 24, 32, 64 and any range derived from these values. In another aspect, the precision for the A/D converter is at least 20 bits. The data obtained can be transmitted to the MCU 400 through an SPI interface, where they can be stored in memory and subsequently transmitted through the first telemetry unit interface to the remote telemetry unit for analysis of the particular analyte levels. Thus, in the above-exemplified glucose sensor, the DAU can, for example, perform the task of sampling the photo diode currents from the 32 photodiodes depicted in FIG. 1. The 32 photodiode array provides 32 channels of the optical sensor, with each channel corresponding to different regions in an NIR spectrum. The DAU 300 can also convert the 32 channels into high precision voltage values, such as for example, 16, 20, or even 24 bit voltage values. An exemplary data acquisition from one photodiode of the 32 photodiode array is illustrated in FIG. 5.

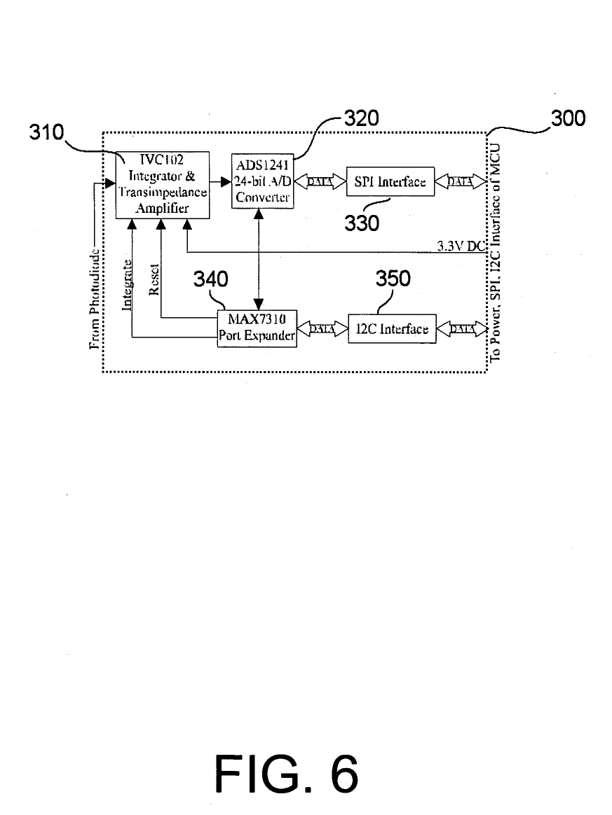

FIG. 6 illustrates an exemplary schematic diagram of a data acquisition unit 300 for one channel of a photo diode. As shown, the data acquisition unit comprises a current integrator 310, such as the IVC102, in communication with a channel of a photodiode array. An analog digital converter 320, such as the ADS1241, is positioned in communication with the integrator 310 and interfaced with the main controller unit via an interface 330, such as an SPI interface. As one of ordinary skill in the art will appreciate, the extension of this schematic diagram to any number of photodiode channels, such as the 32 photodiode array depicted in FIG. 1, is straight forward and can be constructed by one of ordinary skill in the art without requiring undue experimentation. It will also be appreciated by one of ordinary skill in the art that due to the possible limitation of the number of I/O pins on a microcontroller of the Main Controller Unit, port expanders 340 can also be used to generate control signals for the integrator 310. For example, each optional port expander can provides as many as 8 extra I/Os and can also be controlled by the microcontroller of the Main controller unit through an I²C interface 350.

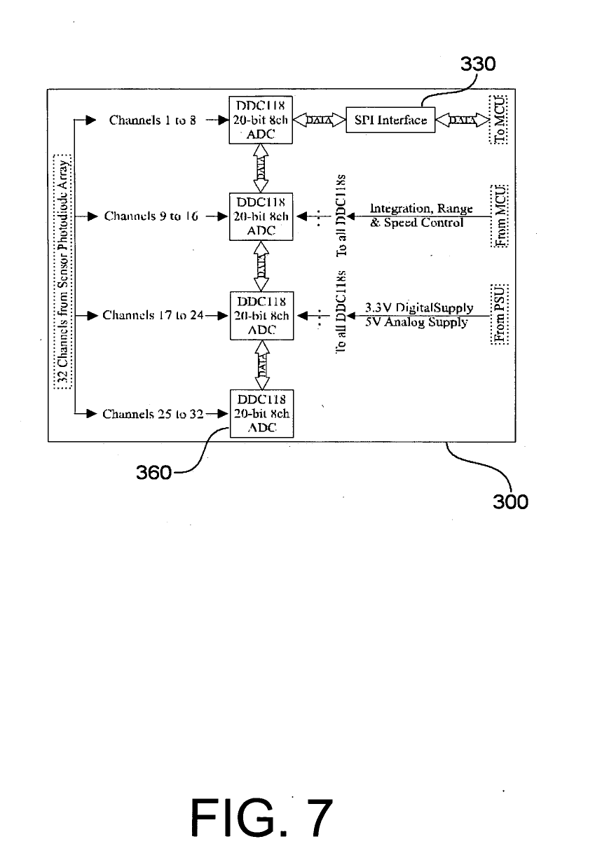

In an alternative aspect, and as depicted in the schematic diagram of FIG. 7, the data acquisition unit can comprise one or more current integrating analog to digital converters 360, such as the Texas Instruments DDC118. According to this aspect, the photo detector current from a photo diode in the analyte sensor can be converted to a voltage by the current integrating analog to digital converter. As one of skill in the art will appreciate, the photo detector current will depend, in part, on the responsivity of the particular photo detector used. Thus, as responsivity of the photo detector is increased, the photo detector current will also increase. In one aspect, a photo detector current will typically be of the order of 10 nA. If a photo detector current is not within the measurable range of an analog to digital converter, an appropriately-selected integrating capacitor can be used to adjust the output voltage of the amplifier to a level that is within the measurable range of the analog to digital converter. To this end, the integrating capacitor needed, will depend on the particular level of the photocurrent and the measurable limits of the analog to digital converter. One of skill in the art will readily be able to optimize the integrating capacitor gain without requiring any undue experimentation. In one aspect, an exemplary integrating capacitor will be 3, 12.5, 25, 37.5, 50, 62.5, 75, or 82.5 pF.

The DDC 118 is an exemplary and commercially available current-integrating analog to digital converter that can be used with a photo diode as described herein. The DDC 118 has integrating capacitors along with a field effect transistor (FET) op-amp which can provide precision voltage corresponding to a particular photo diode current. The signal level can be varied to a desired level by varying the integrating capacitance values and integration times. The DDC 118 can periodically sample and convert to a digital value the integrated current from the photo diode and the resulting value can be stored in the memory of the microcontroller of the Main Controller Unit.

Once again, the extension of the schematic diagram of FIG. 7 to any number of photodiode channels, such as the 32 photodiode array depicted in FIG. 1, is straight forward and can be constructed by one of ordinary skill in the art without requiring undue experimentation. It will also be appreciated by one of ordinary skill in the art that due to the possible limitation of the number of I/O pins on a microcontroller of the Main Controller Unit, port expanders can also be used to generate control signals for the current-integrating analog to digital converter. Each optional port expander can provides as many as 8 extra I/Os and can also be controlled by the microcontroller of the Main controller unit through an I²C interface.

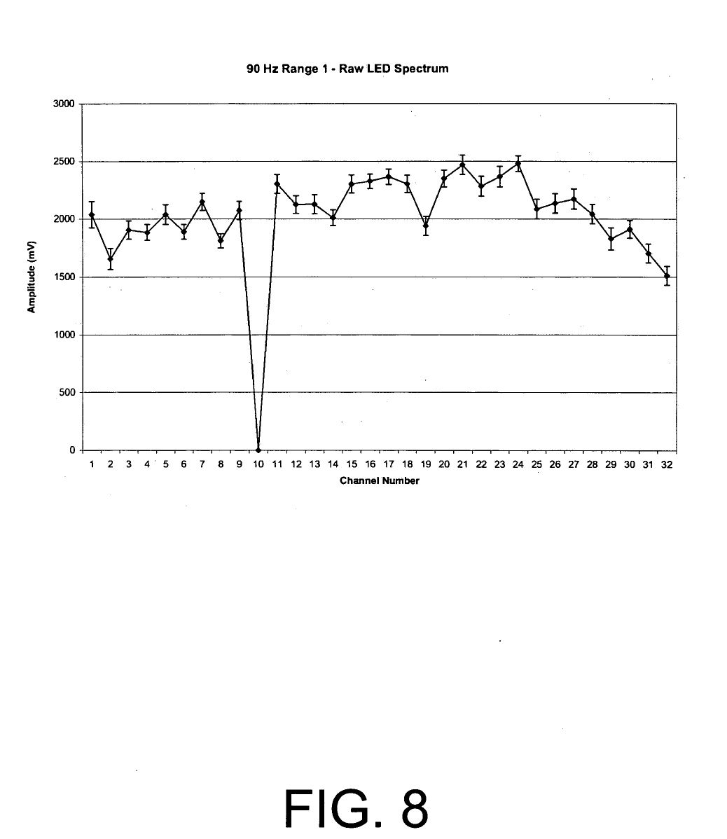

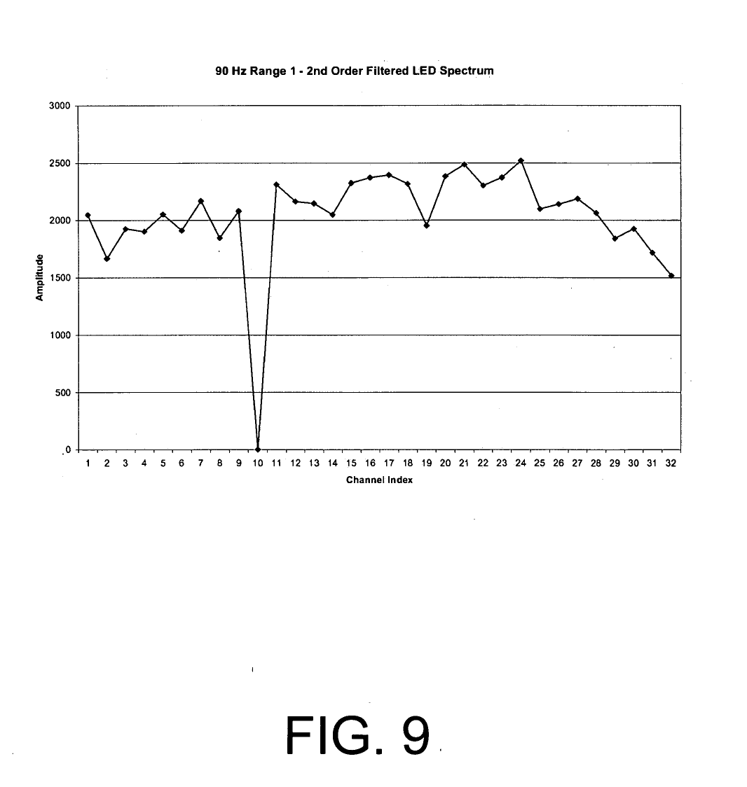

FIGS. 8 and 9 illustrate exemplary sampled absorption data indicating normalized infrared absorption spectra for a representative glucose containing solution. As depicted, each normalized data point corresponds to the data generated by each channel of a 32 channel photodiode array. The particular data sets were obtained from a current integrating digital analog converter, as described herein, using an exemplary 90 Hz sampling frequency, alternating with 2.5 ms of integration with the infrared LED on and 2.5 ms with the LED off. FIG. 8 indicates raw data obtained from the digital analog converter and FIG. 9 indicates the same data after having been filtered with a digital filtering algorithm designed to increase the signal to noise ratio. These exemplary data point are further indicative of the data which can be time stamped and stored in the main controller unit of the electronic support system and transmitted to the remote telemetry unit for further evaluation.

The electronic support system 200 can further comprise a Power Supply Unit 500 that can provide a regulated power supply to one or more modules and/or components of the electronic support module. It should be understood that the power supply unit can be configured to provide any desired level of regulated voltage, depending on the operational requirements of the individual components present within the analyte sensor and electronic support unit. For example and without limitation, in one aspect the power supply can provide a regulated voltage in the range of from approximately 1.0V to approximately 5.0 volts, including voltages of 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.5, 4.6, 4.7, 4.8, 4.9 and any range derived from these values. In another aspect, the power supply provides a regulated voltage ranging from approximately 3.3 V −5V to one or more modules and/or components of the electronic support module.

Additionally, the power supply unit 500 can also provide power for recharging the batteries. Thus, in one aspect, the PSU 500 can be constructed and arranged to comprise an external remote charger unit 510 and an internal inductive power unit 560. According to this aspect, power can be transmitted electromagnetically by the remote charger unit (RCU) 510 to the inductive power unit (IPU) 560 using transcutaneous inductive coupling.

The IPU 560 can supply regulated power to one or more of the units of the internal module. Again, it should be understood that the internal power unit can be configured to provide any desired level of regulated voltage to the internal module depending on the operational requirement of the internal module. In one aspect, the internal power unit can provide regulated voltage in the range of from approximately 1.0V to approximately 5.0 volts, including voltages of 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.5, 4.6, 4.7, 4.8, 4.9 and any range derived from these values. In another aspect, and without limitation, the internal power unit power supply can provide a regulated voltage ranging from approximately 3.3 V −5 V to the internal module.

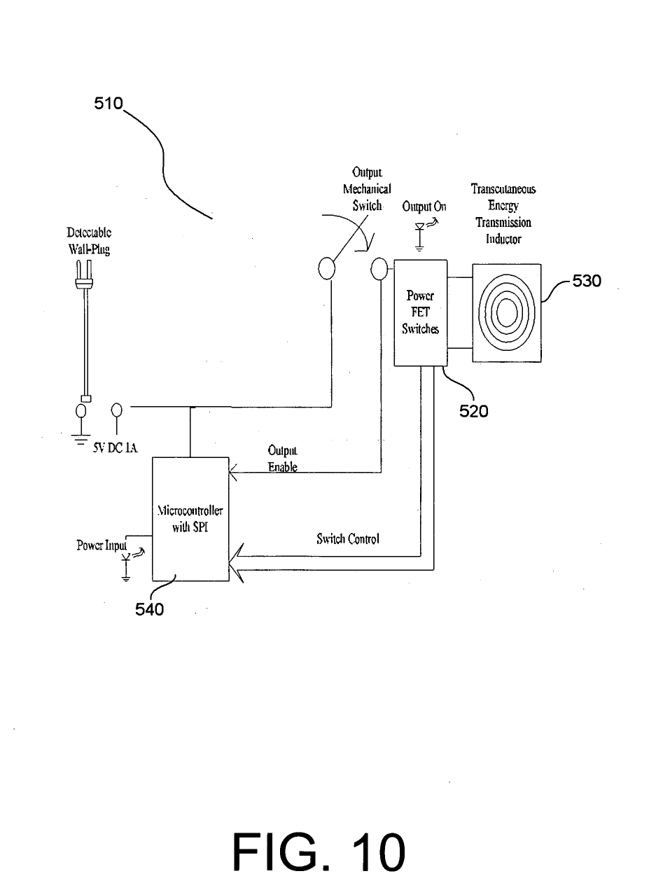

In one aspect, the IPU power source is comprised of two or more battery packs 580, with each pack containing a pair of rechargeable batteries. According to the exemplified aspect in which the IPU supplies a regulated 3.3 V to the internal module, the pair of rechargeable batteries can be, for example, 1.2 V NiMH batteries having 1600 mAh capacity and being connected in series. In use, at any given time a first battery pack can source electrical power to the internal module while the IPU can recharge the second or plurality of second battery packs. The remote charging unit or RCU can also facilitate the charging of the batteries by using transcutaneous inductive coupling through the use of FET switches 520 and a transcutaneous energy transmission inductor 530. The FET switches can generate square waveforms and can be turned ON and OFF alternatively by a microcontroller 540. A schematic of an exemplary RCU 510 is shown in FIG. 10.

The switching rate of the FET's can in one aspect correspond to the optimal transmission frequency of the inductive power unit. To this end, the optimal FET switch rate can be obtained by one of ordinary skill in the art without any undue experimentation. To this end, in the exemplified aspect set forth herein, the optimal switch rate for the FET's can be approximately 4.7 kHz.

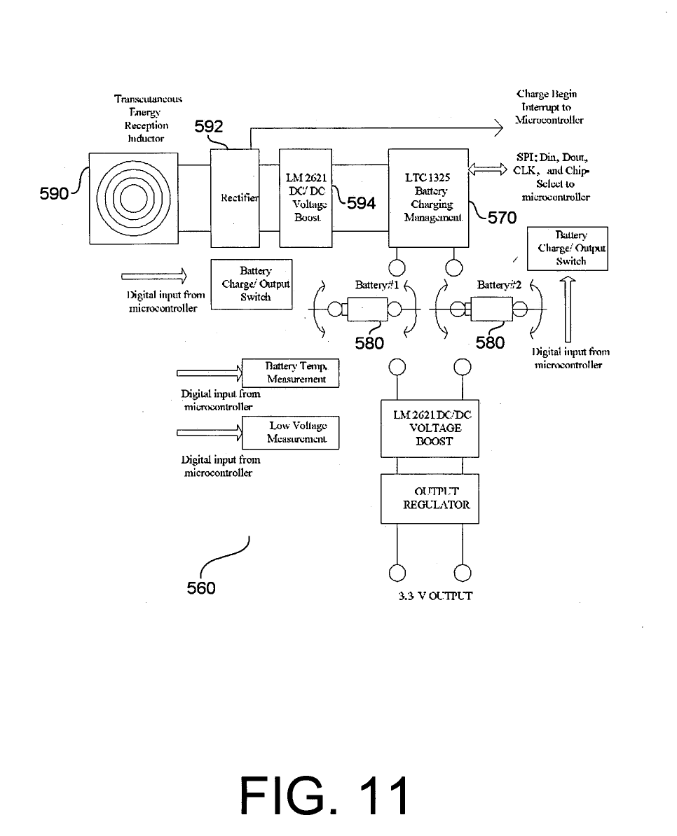

In an exemplary aspect, the IPU 560 can comprise the Linear Technology LTC1325 battery management integrated circuit 570. The LTC1325 is capable of charging a NiMH, Li-ion, and NiCd rechargeable batteries. It is also capable of measuring and/or monitoring battery voltage, battery temperature and/or ambient temperature thereby providing battery status data. The IPU can also comprise its own microcontroller that supervises the LTC1325 through a serial port interface. In use, a fully charged battery pack can have any desired voltage, such as, for example, a voltage of approximately 2.5 V. If needed according to the voltage requirements of the particular system, this voltage can be boosted using an integrated voltage booster circuit and then supplied to a voltage regulator to output a desired voltage, such as, for example, 3.3 V as exemplified above. Accordingly, in one aspect, the IPU is capable of supplying any desired regulated voltage at any desired current load, such as, for example, 700 mAh, in order to power any device within the electronic support unit. A schematic of an exemplary IPU is shown in FIG. 11.

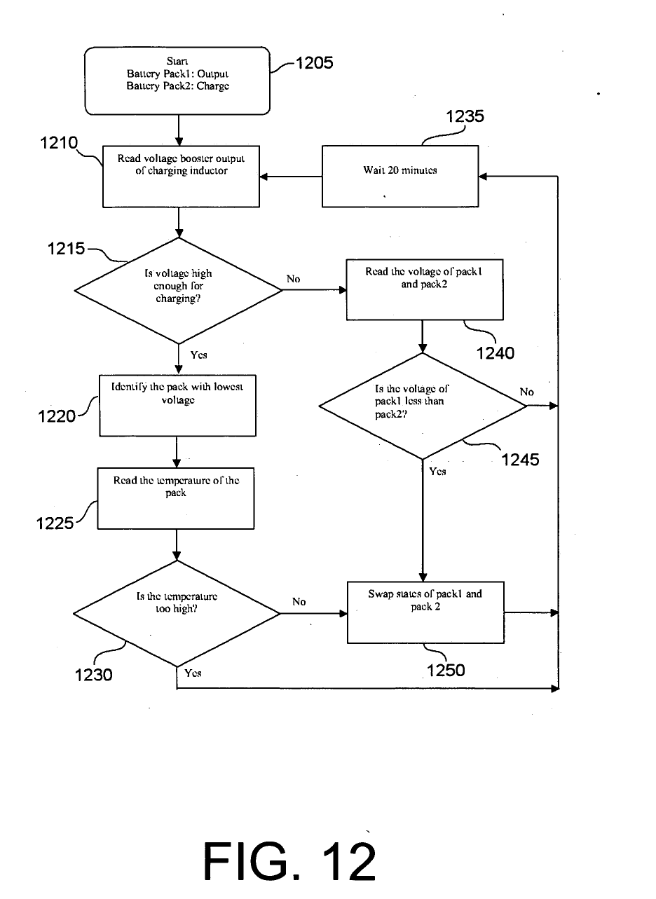

In order to receive the transmitted inductive power, the IPU can use an inductor 590 that is similar or identical to the one used for the RCU. In use, the received waveform can be rectified by a rectifier 592 and fed to a voltage booster unit 594, which boosts the voltage to a desired voltage, such as, for example, approximately 5 V. This voltage can then be used to charge a battery pack 580 and to power a battery management integrated circuit 570 such as the LTC 1325 battery management integrated circuit described above. The microcontroller of the IPU can also communicate with the battery management circuit and, based upon the varying state of the battery, determine which phase of the charging cycle to enter. Exemplary determinations that can be performed by the IPU microcontroller are depicted in a flow chart as illustrated in FIG. 12. As illustrated, the IPU microcontroller can switch to a charging mode when it detects transmission of power. If no power is being transmitted, the battery pack with lower voltage can be switched to the charging mode and the other battery pack can drive the system. Additionally, while the battery is being charged, the battery temperature can also be monitored in order to prevent overheating during the charge cycle.

More specifically, as exemplified in FIG. 12, at block 1205 a first internal battery pack can provide power to the primary module in an output state while a second battery pack can receive a charge from the RCU in a charge state. For the purpose of the exemplified system the method begins with battery pack one in the output state and battery pack two in the charge state. At block 1210 a voltage booster output of the charging inductor is read. The system then proceeds to perform a check at block 1215 to determine if the voltage read is high enough for charging. If the system determines that voltage is high enough for charging, the system proceeds to block 1220, to identify the battery pack with lowest voltage. Then at block 1225, the system reads the temperature of the pack. At block 1230, the system can perform another check to determine if the temperature is too high. If the system determines that the temperature is too high, the system proceeds to block 1235 and can stand by for a predetermined period of time, such as for example 20 minutes. After the predetermined period of time has lapsed, the system returns to block 1210.

If at block 1215 the voltage booster output is not high enough for charging, the system can then proceed to block 1240. At block 1240, the system can read the voltage of battery pack one and battery pack two. Then, at block 1245, the system performs a check to determine the relative voltages of the battery packs, i.e., if the voltage of battery pack one is less than the voltage of battery pack two. If the voltage of battery pack one is less than the voltage of battery pack two the system proceeds to block 1250 and swaps the states of battery packs one and two. If at block 1245 the voltage of battery pack one is not less than the voltage of battery pack two, the system proceeds to block 1235 and can standby for a predetermined period of time before returning to block 1210.

The electronic support system further comprises a telemetry unit 600 or (TU) that can provide a wired or wireless interface between the internal module and the external module. Examples of wireless telemetry connections can include RF, Infrared, 802.xxx, satellite, cellular, and the like. The external module can in one aspect be integrated into a user's personal computer or PDA. Alternatively, the external module can also be a stand alone device. In one aspect, RF telemetry can enable reliable transmission of sensor data on a full-duplex wireless link from the mobile implanted sensor to an external base station. Data can then be collected and sent as packets using a radio protocol that incorporates error detection in order to ensure data accuracy. These packets can also be transmitted to the receiver in any desired frequency, such as, for example, in five minute intervals.

The telemetry unit also comprises a receiving unit that is capable of receiving the data, acknowledging the receipt of valid data, decoding data, and checking for transmission errors. The receiving unit can be interfaced to a PC based system, which can also be integrated into an internet Web based application that can permit local and or remote data analysis by the patient and/or one or more medical health professionals.

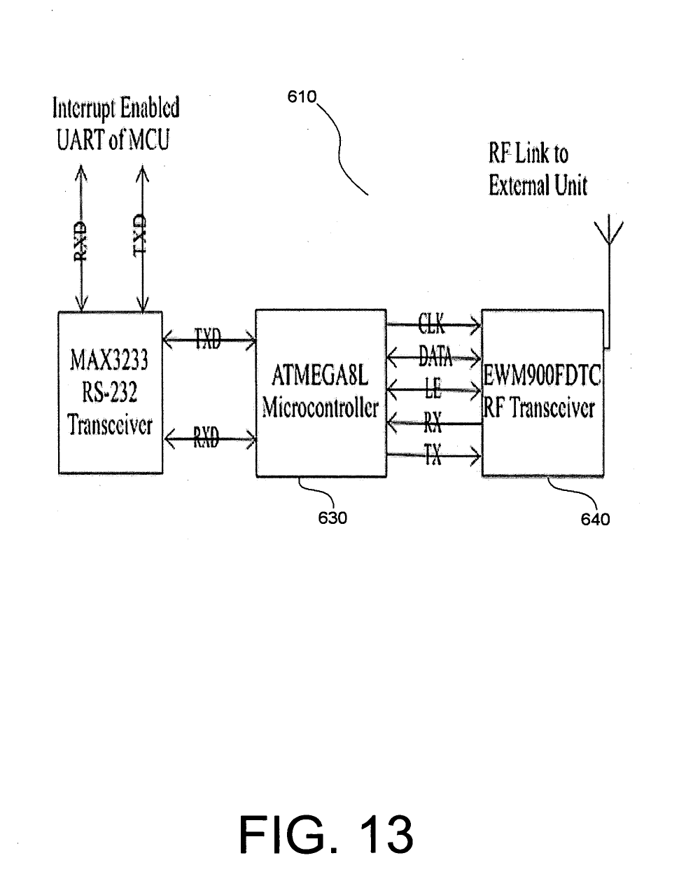

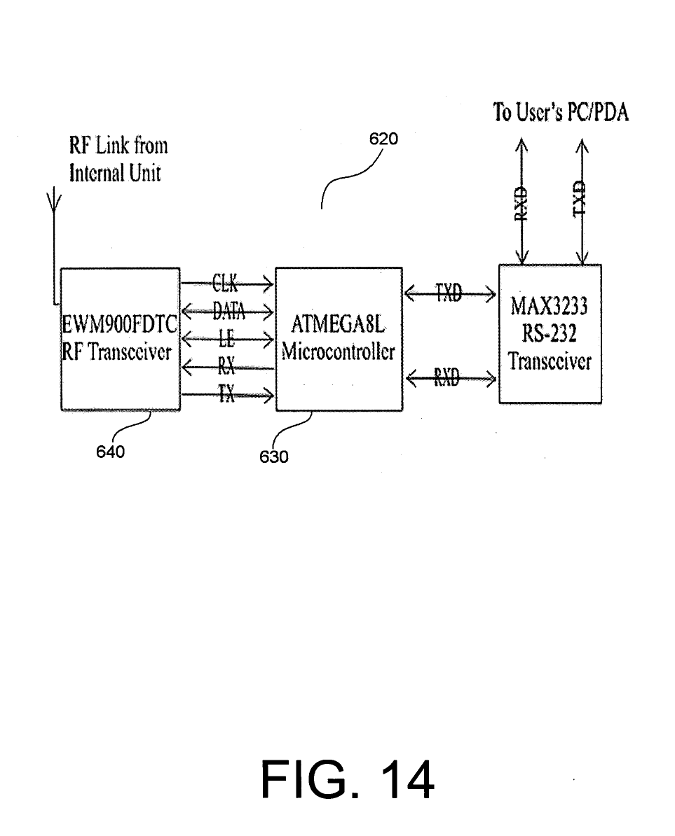

The RF telemetry system can also comprise a first internal or primary telemetry unit 610 which forms a part of the primary module. A remote telemetry unit 620 can also be provided and can be integrated into the remote or external module. Schematic diagrams of an exemplary internal 610 and external telemetry unit 620 are illustrated in FIGS. 13 and 14 respectively. The internal telemetry unit can be similar to or the same as the external unit but is powered by the rechargeable battery power supply. The TU can also be interfaced to both the main controller unit and the power supply unit through conventional interrupt driven protocols.

As will be appreciated upon practicing the invention disclosed herein, the external telemetry unit can enable user access to data through a base station. The external telemetry unit can therefore comprise a microcontroller based system and an RS-232 transceiver. To this end, the block diagram shown in FIG. 14 illustrates an exemplary external telemetry unit comprised of an Atmel Atmega8L AVR microcontroller 630 interfaced to a radio transceiver EWM-900-FDTC 640 through a 3-wire serial interface. If desired, an antenna input can act as the transmitting and the receiving conductor. In one aspect, the antenna input has an impedance of approximately 50 ohms. Digital signals can also be sent to the RF transmitter through the 3-wire serial interface and subsequently converted to radio signals using FM/FSK modulation and then transmitted using the antenna.

The internal telemetry unit is capable of sending sensor data to the external telemetry unit and can also be configured to wait for acknowledgments from the external unit. The internal unit can, in one aspect, transmit 24-bit sensor data along with time stamp information, 16-bit CRC and protocol overhead to the external unit. The radio signals transmitted from the internal telemetry unit can then be received by the external unit through an antenna and converted to digital signals compatible with the CMOS levels for the microcontroller using I/Q demodulation. The received data can also be sent through the UART to a PC or PDA and made accessible to the user. Depending on the choice of components used in the telemetry unit, baud rates of at least 9600 can be used for the data transmission described above. In another aspect, the baud rate can be at least 14400, at least 19200, at least 38400, at least 56000, at least 128000, or at least 256000. To this end, any baud rate capable of providing the data transmission described above can be used in accordance with the present invention.

In another aspect, the internal telemetry unit can be configured to communicate with a remote web server via a network connection, such as over the Internet. The network connection can be, for example, a wired or wireless connection. Examples of wireless connections can include RF, Infrared, 802.xxx, satellite, cellular, and the like. Still further, the internal telemetry unit can be configured to communicate by any one or more of the foregoing exemplary wired or wireless connections. For example, a primary module of the instant invention can be configured to connect to any available 802.xxx connection and transmit sampled biological data to a remote server. Additionally, the sampled data can be encrypted or decrypted as needed. When the primary module is not in range of an available 802.xxx connection, the telemetry can be programmed to automatically switch to a subsequently available communication network.

As described, the ESU can be constructed and arranged to operate continuously and unobtrusively for extended durations with minimal or even no user intervention. Owing to the conditions and the environment in which the sensor and ESU operate, as stated above, in one aspect, a battery based power supply capable of remote charging can be used. It will be also be appreciated upon practicing the present invention that data loss, which can occur when, for example, a user is out of communicable range from the base station for an extended period of time, can be prevented by features implemented in firmware. For example, the ESU can be configured to operate at a relatively low power supply voltage, such as 3.3 V, for reduced power consumption. To this end, a power supply according to this aspect can typically provide more than 24 hours of continuous energy in between successive battery recharge cycles at constant maximum discharge current of, for example, approximately 100 mA. Further, a low power operation mode or sleep mode can be supported as described above in order to conserve the battery energy when the analyte data is not being sampled.

Exemplary and non limiting system specifications concerning data memory capacity, power supply voltage, battery capacity, sampling rate, and data transfer rate are listed in Table 2 below for one aspect of the instant invention.

TABLE 2

| Specifications | Units | Target Value |
|---|---|---|
| Memory Capacity | kB | 32 |
| Power Supply | V | 3.3 |
| Battery Capacity | mAh | 3200 |
| Sampling Rate (max) | Hz | 15 |
| Data Transfer Rate | kbps | 13 |
| Serial Interface | Type | SPI, I²C, RS-232 |

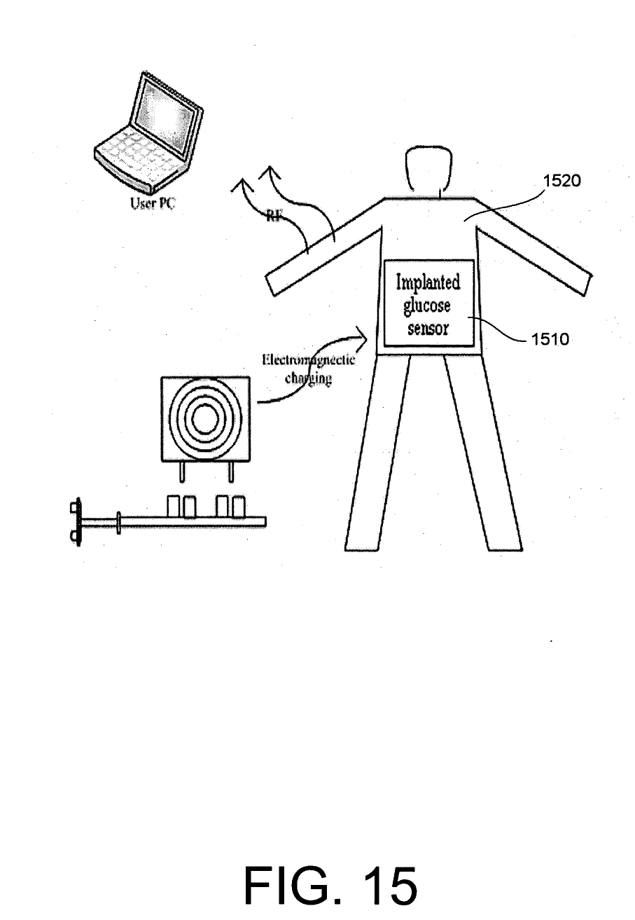

FIG. 15 illustrates an exemplary physical implementation of an analyte sensor 1510 into a test subject 1520. In use, the sensor can be implanted in the subcutaneous tissues of, for example, the human body. The ESU can enable the sensor to operate for months with minimal user intervention. During operation, the interstitial fluid from subcutaneous space can be sampled through an embedded ultra filtration probe and can then enter into a micro fluidic chamber, which can be physically isolated from the biological environment. If, for example, glucose is the analyte under investigation, then the sample can be carried to an optimized spectrometer cell, where a 16 cm⁻¹ resolution near infrared spectrum is collected over a spectral range of from approximately 4600 to approximately 4200 cm⁻¹ (2.17-2.38 μm). The uniqueness of the glucose spectrum in this waveband is illustrated in FIG. 2. The concentration of the glucose can then be obtained from direct analysis of the collected absorbance data in the selected waveband. As one of ordinary skill in the art will appreciate, the spectral range illustrated above is optimized for use in connection with glucose. Thus, the desired spectral range will be dependent upon the particular analyte under investigation.

The electronic support unit described and disclosed herein can be used in a variety of applications. As such, in another aspect, the present invention provides a method for performing any one or more of the applications disclosed herein, wherein the method further comprises utilization of an ESU as described herein. For example, the ESU can be used in connection with analyte concentration measurement, analysis, data logging, storage, and/or transmission. In one aspect, the analysis of an analyte concentration in a test subject can be accomplished by using an order derivative of the absorption data collected by the data acquisition unit, including zero order, optionally combined with other forms of data pre-processing. Any statistical technique may be used to derive the primary calibration algorithm, for example, which should not be considered limiting in any way, simple linear regression, multiple linear regression and multivariate data determination. Examples of multivariate data analysis, which should not be considered limiting in any way, are principle component analysis, principle component regression, partial least squares regression, and neural networks. Examples of data pre-processing, which should also not be considered limiting in any way, can include smoothing, deriving a first higher order derivative of absorbance, interpolation of absorbance, multiplicative scatter correction, photometric correction, and data transformation, such as Fourier Transform.

In one aspect, a computing apparatus for computing and analyzing the analyte concentration from the data transmitted to the external telemetry unit can comprise a processor such as a microprocessor, a hybrid/software system, controller, computer, neural network circuit, digital signal processor, digital logic circuits, or an application specific integrated circuit, and memory. The computing apparatus can be electronically coupled to the data received by the external telemetry unit and can contain circuits programmable to perform mathematical functions such as, for example, waveform averaging, amplification, linearization, signal rejection, differentiation, integration, network or fuzzy logic, addition, subtraction, division, multiplication, and the like where desired.

In an alternative aspect, and apart from assisting a user, physician or other medical professional in monitoring analyte levels, such as blood glucose levels of patients in real time, the sensor unit comprising an ESU as described herein can in another aspect be used as a feedback element in an insulin delivery system, where, for example, the entire system can function as an artificial pancreas. Thus, in another aspect, the present invention provides an artificial biological delivery system comprising an ESU as described herein.

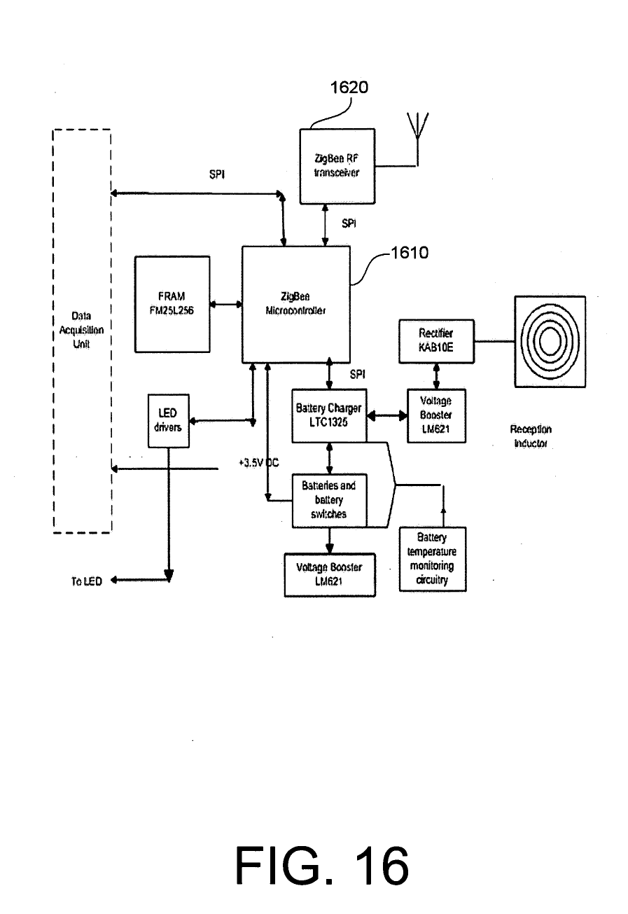

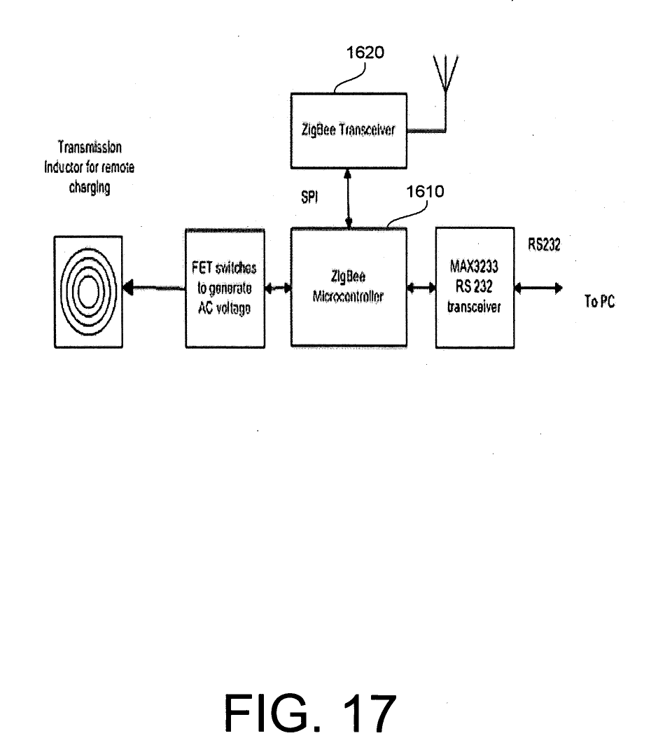

In still another aspect, the electronic support system can be adapted for use with a plurality of other sensor units involving the measurement of biological data. For example, individual sensor units can be adapted to function as nodes of a larger network through the use of the ESU's adaptable telemetry unit. For example, use of a ZigBee 802.15.4 protocol based microcontroller 1610 and transceiver 1620 can be used in the instant invention. IEEE 802.15.4 is a wireless technology protocol standard targeted at home networking and sensor networks and, when used, can permit up to, for example, 255 nodes to exist in one network. It is an ultra low power technology with relatively low system hardware requirements and can provide up to 250 kbps of bandwidth. Thus, the use of 802.15.4 technology in the instant invention can provide an ESU having reduced power consumption and increased security in transmissions. Still further, any desired number of such networks could be set up in, for example, a hospital and the nodes (individual sensor units) could all be controlled remotely from a central location. FIGS. 16 and 17 illustrate alternative aspects of the instant invention comprised of components using the 802.15.4 based ZigBee protocol.

In view of the foregoing, it will be apparent to those skilled in the art that various modifications and variations can be made in the present invention without departing from the scope or spirit thereof. As such, other aspects of the present invention will become apparent to those skilled in the art from consideration of the instant specification and practice of the invention disclosed herein.

What is claimed is:

1. An electronic support system for controlling an implanted biological data sensor, comprising:
a) a primary module comprised of: i) a main controller having a data acquisition interface and a telemetry interface; ii) a data acquisition unit in communication with the main controller through the data acquisition interface and configured to receive data transmitted from a biological data sensor; iii) a first telemetry unit in communication with the main controller through the telemetry interface; iv) an internal power unit; and
b) a remote module comprised of a remote charging unit and a second telemetry unit,
wherein the first telemetry unit is configured to communicate with second telemetry unit.

2. The electronic support system of claim 1, wherein the primary module is implantable into a test subject

3. The electronic support system of claim 1, wherein the biological data sensor is a biological analyte sensor.

4. The electronic support system of claim 3, wherein the analyte is glucose.

5. The electronic support system of claim 1, wherein the main controller comprises a microcontroller and a memory capacity.

6. The electronic support system of claim 5, wherein the memory capacity is capable of storing at least 24 hours of sampled data.

7. The electronic support system of claim 5, wherein the microcontroller is programmed with an instruction set.

8. The electronic support system of claim 5, wherein the microcontroller is an 8 or 16 bit microcontroller.

9. The electronic support system of claim 1, wherein the data acquisition interface comprises a serial peripheral interface.

10. The electronic support system of claim 1, wherein the telemetry interface comprises an RS-232 serial port.

11. The electronic support system of claim 1, wherein the data acquisition unit is configured to receive sample data from a photodiode array.

12. The electronic support system of claim 1, wherein the data acquisition unit comprises a current integrator in communication with the implantable biological sensor.

13. The electronic support system of claim 12, further comprising an analog to digital converter in communication with the current integrator.

14. The electronic support system of claim 1, wherein the data acquisition unit comprises a current integrating analog to digital converter.

15. The electronic support system of claim 14, wherein the analog/digital converter provides digitized voltage values having a level of precision in the range of from 8 bits to 128 bits.

16. The electronic support system of claim 15, wherein the analog/digital converter provides digitized voltage values having a level of precision in the range of from 16 bits to 24 bits.

17. The electronic support system of claim 1, wherein the first telemetry unit comprises a RF transmitter.

18. The electronic support system of claim 17, wherein the RF transmitter is configured to communicate with a second telemetry unit according to IEEE 802.15.4 wireless protocol.

19. The electronic support system of claim 1, wherein the internal power unit can supply a regulated voltage to one or more components of the primary module in the range of from 3 volts to 4 volts.

20. The electronic support system of claim 1, wherein the internal power unit can be electromagnetically charged from the remote charging unit by inductive coupling.

21. The electronic support system of claim 2, wherein the primary module is implantable into the subcutaneous tissue of a test subject.

22. The electronic support system of claim 1, wherein the second telemetry unit comprises an RF receiver configured to receive data transmitted from the first telemetry unit.

23. The electronic support system of claim 1, wherein the remote module can be positioned a distance of up to 200 feet from the primary module.

24. A method for monitoring a concentration of an analyte in a test subject; comprising the steps of:
sampling electromagnetic absorption data of an analyte in a test subject with an implantable analyte sensor;
transmitting the sampled data in digitized format to a main controller unit wherein at least a portion the digitized data is time stamped and stored as processed data;
transmitting at least a portion of the processed data to a remote telemetry module; and
determining the concentration of the analyte in the test subject at a predetermined time from at least a portion of the processed data received by the remote telemetry module.

25. The method of claim 24, wherein the analyte is glucose.

26. The method of claim 24, wherein at least a portion of the processed data is stored for a predetermined period prior to being transmitted to the remote telemetry unit.

27. The method of claim 26, wherein at least a portion of the processed data is stored for a period of up to 24 hours prior to being transmitted to the remote telemetry unit.

28. The method of claim 24, wherein the electromagnetic absorption data is obtained from a photodiode array.

29. The method of claim 24, wherein the processed data is transmitted to the remote telemetry module according to IEEE 802.15.4 wireless protocol.

30. The method of claim 24, wherein the digitized data comprises a level of precision in the range of from 16 bits to 24 bits.

31. An insulin delivery system comprising the electronic support system of claim 1.
